(19) United States
(12) Patent Application Publication
(10) Pub. No.: US 2020/0043613 A1
(43) Pub. Date: Feb. 6, 2020

(54) CAPSULE ENDOSCOPY IMAGE REVIEW AND QUALITY CONTROL SYSTEM AND CONTROL METHOD THEREOF

(71) Applicant: Ankon Technologies Co., LTD., Wuhan (CN)

(72) Inventors: HAO ZHANG, Wuhan (CN); NA ZENG, WUHAN (CN); Xinhong WANG, Wuhan (CN)

(21) Appl. No.: 16/529,659

(22) Filed: Aug. 1, 2019

(30) Foreign Application Priority Data
Aug. 1, 2018 (CN) 201810864341.1

(51) Int. Cl. G16H 50/50; G16H 40/63; G16H 30/20; G16H 50/20; G16H 30/40; H04L 29/06; G06N 3/08; G06T 7/00; G06T 7/73; A61B 1/04; A61B 1/00

(52) U.S. Cl. CPC G16H 50/50; G16H 40/63; G16H 30/20; G16H 50/20; G16H 30/40; H04L 63/0428; H04L 67/12; G06T 7/0012; G06T 7/73; A61B 1/041; A61B 1/00158; A61B 1/00016; A61B 1/00055; G06N 3/08

(57) ABSTRACT

The present invention provides a capsule endoscopy image review and quality control system and control method thereof. The capsule endoscopy image review and quality control system include a capsule endoscopy data acquisition system and a local server in communication with the capsule endoscopy data acquisition system. The capsule endoscopy data acquisition system includes a capsule endoscope, an external magnetic field device for controlling movement and/or rotation of the capsule endoscope, and a controller in communication with the capsule endoscope and the external magnetic field device. The local server includes a digestive tract position identification module and a corresponding matching module for quality control process, wherein the corresponding matching module for quality control process and the digestive tract position identification module are in communication with the controller.

### CROSS-REFERENCE OF RELATED APPLICATIONS

The application claims priority to Chinese Patent Application No. 201810864341.1 filed on Aug. 1, 2018, the contents of which are incorporated by reference herein.

### FIELD OF INVENTION

The present invention relates to a medical device, and more particularly to a computer-assisted capsule endoscopy image review and quality control system and control method thereof.

### BACKGROUND

Magnetic capsule endoscopy is a new type of medical device for digestive tract examination, but requirement for an operator to use such a device is very high, as it is easy to cause missed detection and false detection if operation standards are not properly followed.

An existing computer-aided diagnosis system comprises a message server cluster and work nodes of a plurality of computer-aided diagnosis servers, which obtains processing results by acquiring streaming data and performing real-time parallel stream processing. However, by using this method, the computer-aided diagnosis results are stored in the database and cannot be pushed to the examiner in real time to realize quality control of the examination process. The connection mode between the input device and the computer-aided diagnosis system in the method fails to effectively solve the problem of real-time response of the computer-aided diagnosis results. The method also does not provide an automatic matching method between the input device and the computer-aided diagnosis system, which requires manual configuration when the system is configured.

An existing medical imaging system is developed using the DICOM3.0 protocol. The system adopts a centralized client/server structure in combination with the TCP/IP protocol and provides storage for patient images and general information retrieval. However, the solution does not realize the function of identifying the images, and therefore fails to feed back the examination results during operation in real time. Either, the solution does not provide a terminal for image input or an interface for device access, and therefore cannot realize access of image acquisition equipment.

It is necessary to provide a capsule endoscopy image review and quality control system and control method thereof to solve the above technical problems.

### SUMMARY OF THE INVENTION

The present invention provides a computer-assisted capsule endoscopy image review and quality control system to automatically identify digestive tract regions and lesions, and automatically match standard operating procedures of the magnetic capsule endoscopy system, and reminds the operator in real time to operate the system, so as to achieve the purpose of quality control of the capsule endoscopy.

In one embodiment, the present invention provides a capsule endoscopy image review and quality control system comprising a capsule endoscopy data acquisition system and a local server in communication with the capsule endoscopy data acquisition system, wherein the capsule endoscopy data acquisition system comprises a capsule endoscope, an external magnetic field device for controlling the movement and/or rotation of the capsule endoscope, and a controller in communication with both the capsule endoscope and the external magnetic field device; and wherein the local server comprises a digestive tract position identification module and corresponding matching module for quality control process, wherein corresponding matching module for quality control process and the digestive tract position identification module are in communication with the controller.

In one embodiment, the digestive tract position identification module comprises an image data screening module, a digestive tract region identification module, a digestive tract lesion identification module and a position identification module, wherein the image data screening module screens image data acquired from the capsule endoscopy data acquisition system to remove unclear, over-bright or over-dark images; wherein the digestive tract region identification module comprises a digestive tract anatomical region identification algorithm for identifying anatomical regions of the digestive tract according to the screened images; wherein the digestive tract lesion identification module comprises a digestive tract lesion identification algorithm for identifying positive lesions of the digestive tract; and wherein the position identification module identifies the relative position of the digestive tract where the capsule endoscope is located and the lesions at current position by analysis of anatomical region of the digestive tract and data from sensors inside the capsule endoscope, and sensors inside the capsule endoscope comprise an acceleration sensor, a gyroscope, a TOF distance sensor and a magnetic field sensor. As a further improvement of the invention, the corresponding matching module for quality control process comprises a preset operation quality control model corresponding to the digestive tract position and/or the lesion information.

In one embodiment, the corresponding matching module for quality control process comprises a preset operation quality control model corresponding to the digestive tract position and/or the lesion information.

As a further improvement, the local server and the capsule endoscopy data acquisition system are connected through a local area network, or are directly connected through a switch, a router, and a networking cable.

As a further improvement, the capsule endoscopy reading and quality control system further comprises a cloud server in communication with the local server.

As a further improvement, the cloud server is connected to the local server via the Internet or the intranet.

As a further improvement, the connection between the cloud server and the local server is encrypted.

As a further improvement, the service architecture of the cloud server includes web service, application service, cloud storage, load balancing and message queue service and deep learning service cluster.

In another embodiment, the present invention provides a capsule endoscopy image review and quality control method, comprising a capsule endoscopy data acquisition system transmits image data and sensor data to a local server; the local server achieves quality control process based on the image data and the sensor data through a digestive tract position identification module of the local server and corresponding matching module for quality control process of the local server, wherein the digestive tract position identification module processes the image data and the sensor data, and the corresponding matching module for quality control process generates quality control operations according to the processing results of the digestive tract position identification module, and returns lesion identification results and the quality control operations to the capsule endoscopy data acquisition system.

In one embodiment, wherein the processing flow of the digestive tract position identification module on the image data and the sensor data is that an image data screening module of the digestive tract position identification module pre-processes the image data received from the capsule endoscopy data acquisition system to remove unclear, over-bright or over-dark images; a digestive tract anatomical region identification algorithm of the digestive tract position identification module identifies anatomical regions of the digestive tract according to the screened images, and a digestive tract lesion identification algorithm of the digestive tract position identification module identifies positive lesions in the digestive tract; and a position identification module of the digestive tract position identification module identifies the relative position of the digestive tract where the capsule endoscope is located and the positive lesions at current position by analysis of anatomical region of the digestive tract and sensor data from sensors inside the capsule endoscope, wherein the sensors inside the capsule endoscope comprises an acceleration sensor, a gyroscope, a TOF distance sensor and a magnetic field sensor.

As a further improvement, the digestive tract position identification module employs a heterogeneous computing technology of CPU+GPU or CPU+FPGA in the image processing method.

As a further improvement of the invention, the quality control process of the local server comprises that the digestive tract position identification module identifies the digestive tract position information and lesion information by processing the image data and the sensor data, and sends the identified information to the corresponding matching module for quality control process; the corresponding matching module for quality control process generates a corresponding quality control operation code according to digestive tract position information, lesion information and an operation quality control model, and transmits the quality control operation code to the capsule endoscopy data acquisition system, wherein the quality control operation code contains the information about the operation performed currently; and after receiving the quality control operation code, the capsule endoscopy data acquisition system determines whether the capsule endoscope reaches a threshold for the digestive tract position segment, if the threshold is reached, specific contents of the quality control operation code is presented to an operator, and if the threshold is not reached, a quality control identification is recorded.

As a further improvement of the invention, wherein the quality control process further comprises: when the digestive tract position identification module identifies a region of the digestive tract, the region is highlighted on a simulated digestive tract 3D model on the display of the capsule endoscopy data acquisition system, and when the digestive tract position identification module identifies a suspected lesion in the digestive tract, a quality control operation code corresponding to the suspected lesion is presented in a real-time browsing interface of the display.

As a further improvement, the capsule endoscopy image review and quality control method further comprises an automatic configuration method and procedure of the local server and the capsule endoscopy data acquisition system, wherein

the capsule endoscopy data acquisition system sends an IP multi-cast or IP broadcast message to the local area network, and waits for the unicast response from the local server;

if the capsule endoscopy data acquisition system receives message from the local server, the capsule endoscopy data acquisition system records the IP address of the local server and establishes Socket or RPC connection with the local server;

after the connection is established, the configurations of the capsule endoscopy data acquisition system and the local server are synchronized.

As a further improvement, the capsule endoscopy image review and quality control method further comprises remotely updating main control program, image processing algorithm, anatomical region identification algorithm, digestive tract lesion identification algorithm, deep learning model for the local server through a cloud server.

The capsule endoscopy image review and quality control system disclosed herein generates the quality control operations according to the processing results of the digestive tract position identification module through the corresponding matching module for quality control process, and returns the lesion identification result and the quality control operations to the capsule endoscopy data acquisition system. It has provided guidance for the operation of physicians.

### BRIEF DESCRIPTION OF THE DRAWINGS

FIG. 1 shows a schematic view of communication between a capsule endoscopy image review and quality control system and a capsule endoscopy data acquisition system in accordance with a preferred embodiment of the present invention.

FIG. 2 shows a schematic view of quality control process of a local server and the capsule endoscopy data acquisition system in accordance with a preferred embodiment of the present invention.

FIG. 3 shows a schematic view of service contents of a cloud server in accordance with the present invention.

FIG. 4 shows a schematic view of automatic configuration process between the capsule endoscopy image review and quality control system and the capsule endoscopy data acquisition system in accordance with a preferred embodiment of the present invention.

### DETAILED DESCRIPTION

The present invention can be described in detail below with reference to the accompanying drawings and preferred embodiments. However, the embodiments are not intended to limit the invention, and the structural, method, or functional changes made by those skilled in the art in accordance with the embodiments are included in the scope of the present invention.

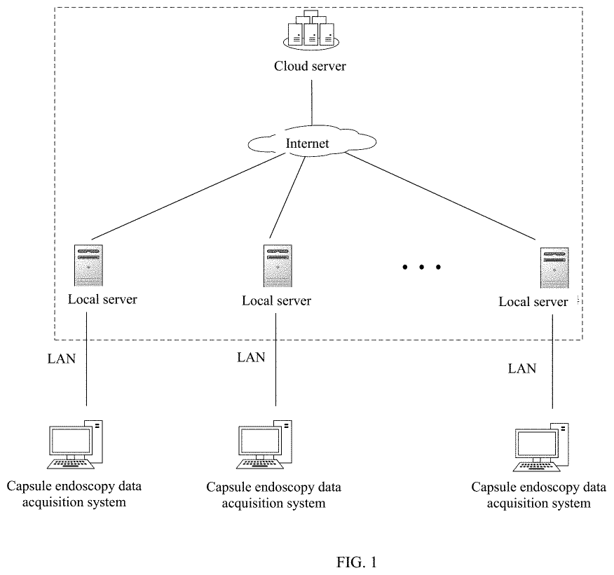

Referring to FIG. 1, a schematic view of a preferred embodiment in accordance with the present invention is shown, wherein a capsule endoscopy image review and quality control system is provided, comprising a capsule endoscopy data acquisition system and a local server communicating with the capsule endoscopy data acquisition system.

The capsule endoscopy data acquisition system generally comprises a capsule endoscope, an external magnetic field device for controlling the movement and/or rotation of the capsule endoscope, and a controller in communication with both the capsule endoscope and the external magnetic field device. The capsule endoscope comprises an image acquisition unit for acquiring image data inside the digestive tract, and sensors for controlling or assisting in determining the posture of the capsule endoscope. The sensors include, but not limited to, an acceleration sensor, a gyroscope, a TOF (Time-of-Flight) distance sensor and a magnetic field sensor.

The capsule endoscopy data acquisition system is used to collect image data of different positions, different angles and different regions in the digestive tract. Other structures and operation modes of the capsule endoscope are implemented by using existing technologies, and do not describe herein.

The local server comprises two functional modules: corresponding matching module for quality control process and a digestive tract position identification module. The corresponding matching module for quality control process is in communication with the digestive tract position identification module. The digestive tract position identification module provides digestive tract position information and lesion information to the corresponding matching module for quality control process.

The local server and the capsule endoscopy data acquisition system are connected through a local area network, or directly connected through a switch, a router, and a networking cable. The configuration of upon connection is simple, plug and play. The capsule endoscopy data acquisition system transmits image data and sensor data to the local server in real time, and the local server returns the lesion identification result and the quality control operations after processing the image data and the sensor data. The image data and sensor data are in JPG, DICOM, PNG, BMP or other formats.

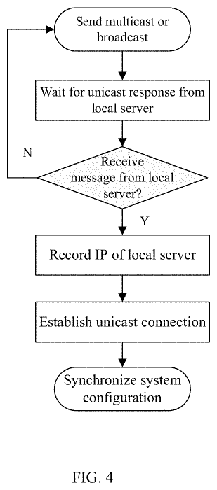

The local server and capsule endoscopy data acquisition system can be automatically configured, and the automatic configuration method and procedure are shown in FIG. 4. Firstly, the capsule endoscopy data acquisition system sends an IP multi-cast or IP broadcast message to the local area network, and waits for a unicast response from the local server. If the capsule endoscopy data acquisition system receives message from the local server, the capsule endoscopy data acquisition system records the IP address of the local server and establishes Socket or RPC connection with the local server. After the connection is established, system configurations of the capsule endoscopy data acquisition system and the local server are synchronized without manual configuration.

Those skilled in the art can understand that multi-cast refers to implementing a point-to-multipoint network connection between a sender and each receiver. Broadcast refers to broadcasting data packets within an IP subnet, and all hosts inside the subnet can receive these data packets. Unicast refers to implementing a point-to-point network connection between the sender and each receiver. In an example of the present invention, the capsule endoscopy data acquisition system transmits IP multicast or IP broadcast messages to the local area network, and the local servers located in the local area network can receive the messages, and when a local server feeds back information to the capsule endoscopy data acquisition system, a point-to-point connection is established between the two.

The digestive tract position identification module is configured for processing image data and employs a heterogeneous computing technology of CPU+GPU or CPU+FPGA in the image processing method to considerably improve image processing speed. The image processing method includes a digestive tract lesion identification algorithm, which can identify the characteristics of positive lesions in the digestive tract and generate a thermodynamic diagram and a bounding box to indicate the location of the lesion. The image processing method also includes an digestive tract anatomical region identification algorithm, which can effectively identify different regions of the digestive tract including esophagus, dentate line, cardia, fundus, greater curvature, lesser curvature, angulus, antrum, pylorus, duodenum, duodenal bulb and descending part, jejunum, ileum, colon and etc.

Specifically, the digestive tract position identification module comprises an image data screening module, a digestive tract region identification module, a digestive tract lesion identification module, and a position identification module that analyzes the relative position of the digestive tract where the capsule endoscope is located and the lesions at current position based on the sensor data. The image data screening module is configured for screening image data acquired from the capsule endoscope data acquisition system to to remove various unclear, over-bright or over-dark images. The digestive tract region identification module comprises the digestive tract anatomical region identification algorithm for identifying anatomical regions of the digestive tract according to the screened image. The digestive tract lesion identification module comprises the digestive tract lesion identification algorithm for identifying positive lesions of the digestive tract.

The processing flow of the digestive tract position identification module on the image data and the sensor data includes: firstly, the image data screening module pre-processes the image data received from the capsule endoscopy data acquisition system to remove various unclear, over-bright or over-dark images; secondly, the digestive tract anatomical region identification algorithm identifies the anatomical region of the digestive tract according to the screened images, and the digestive tract lesion identification algorithm identifies the positive lesion in the digestive tract; thirdly, the position identification module analyzes posture information (current displacement, angle, etc.) of the capsule endoscope based on anatomical region of the digestive tract and sensor data from sensors inside the capsule endoscope, and identifies the relative position of the digestive tract where the capsule endoscope is located and the lesions at current position based on the posture information. The sensors inside the capsule endoscope comprise, but not limited to, the acceleration sensor, gyroscope, TOF distance sensor and magnetic field sensor.

The digestive tract anatomical region identification algorithm according to the present invention can be understood by reference to the algorithm in the Chinese patent application No. 201710267329.8.

The corresponding matching module for quality control process comprises a preset operation quality control model corresponding to the digestive tract position information and/or the lesion information.

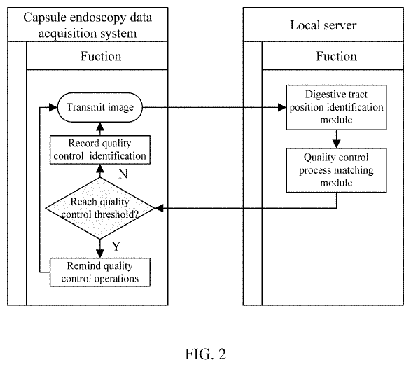

The local server implements quality control process based on the image data and the sensor data through the digestive tract position identification module and the corresponding matching module for quality control process. As shown in FIG. 2, firstly, the digestive tract position identification module identifies the digestive tract position information and the lesion information according to the image data and the sensor data, and sends the identified information to the corresponding matching module for quality control process. Secondly, the corresponding matching module for quality control process generates a quality control operation code according to the digestive tract position information, the lesion information and the operation quality control model, and transmits the quality control operation code to the capsule endoscopy data acquisition system. The quality control operation code contains the information about the operation that should be performed currently. Thirdly, after receiving the quality control operation code, the capsule endoscopy data acquisition system determines whether the capsule endoscope reaches a threshold of the digestive tract position segment. If the threshold is reached, it means that the capsule endoscope has been run to a certain part of the digestive tract and the images are taken, and specific contents of the quality control operation is presented to an operator. If the threshold is not reached, it means that the capsule endoscope has not been run to the certain part of the digestive tract, and a quality control identification is recorded. The quality control identification refers to the number of the quality control operation, and the capsule endoscopy data acquisition system continues to send image data and sensor data to the local server, and repeats the above process.

The quality control process of the capsule endoscopy image review and quality control system is: when the digestive tract position identification module identifies a key region of the digestive tract, the region is highlighted on a simulated digestive tract 3D model on a display device of the capsule endoscopy data acquisition system; and when the digestive tract position identification module identifies a suspected lesion in the digestive tract, the quality control operation code of the suspected lesion is presented to the operator in a real-time browsing interface of the display device.

The capsule endoscopy image review and quality control system further comprises a cloud server in communication with the local server to assist the local server in processing and calculating the image data and the sensor data, etc.

The connection between the cloud server and the local server is: the two can be connected through the Internet or connected through the internal LAN of the enterprise, and on this basis, the encrypted connection such as VPN or SSL can also be used to ensure data security.

The cloud server can remotely update main control program, image processing algorithm, the digestive tract anatomical region identification algorithm, the digestive tract lesion identification algorithm, and deep learning model for the local server. The update is based on existing technology, that is, transferring a new program to the local server to update the old program, and is not described here. In addition, when running data analysis algorithms with a large amount of computation, the local server uploads the preprocessed data to the cloud server for processing, and receives processing results from the cloud server.

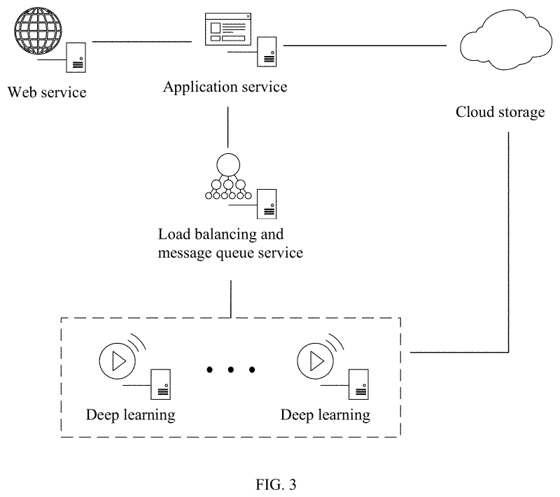

The software service architecture of the cloud server is shown in FIG. 3, comprising web service, application service, cloud storage, load balancing and message queue service and deep learning service cluster. The Web service is used to publish Web services to users of the capsule endoscopy image review and quality control system. The Web service can adopt the CGI gateway, the Apache service or nginx service on Linux system, the IIS service on Windows system, and the Web service supports the http and https protocols. The application service provides application program interface of cloud service storage, image recognition and operation quality control service. The application service also provides the secure encryption authentication interface. The application service supports protocols such as JSON and XML, and supports encryption and token authentication. Cloud storage provides the function of massive data storage. The cloud storage service can be an object storage service, and also can be a distributed relational database, various NoSQL databases, or a Key-Value database. Load balancing and message queue service evenly distributes computing tasks to deep learning clusters. With distributed message queue service, load balancing supports multiple strategies such as round-robin, weight, and traffic ratio, etc. Deep learning service cluster has powerful computing capabilities, supports openMPl (parallel computing library), Apache spark big data analysis platform, tensor computing platform Tensorflow of Google, deep learning library Torch, deep learning platform Theano, deep learning platform MXNet and more platforms, and deploys a variety of deep learning inference models to meet application requirements, such as deep learning models for identifying digestive tract lesions and digestive tract regions.

When the local server is in connection with the cloud server, the local server can send intermediate results of image or data processing to the cloud server. The intermediate results include, but are not limited to, scale-invariant feature transform (SIFT), histogram of oriented gradient (HOG), speed up robust features (SURF), vector or tensor generated by the deep learning convolution computing, and the multidimensional array composed of images. When the local server and the cloud server are unable to communicate or are not authorized, the local server can process the image data directly and send the result to the capsule endoscopy data acquisition system.

Based on the capsule endoscopy image review and quality control system, the capsule endoscopy image review and quality control method of the present invention includes all processes and methods described above, and only some of the methods are described systematically and briefly as follows.

The capsule endoscopy image review and quality control method comprises: the capsule endoscopy data acquisition system transmits image data and sensor data to the local server; the local server implements quality control process based on the image data and the sensor data through the digestive tract position identification module and the corresponding matching module for quality control process. The digestive tract position identification module processes the image data and the sensor data, and the corresponding matching module for quality control process generates quality control operations according to the processing results of the digestive tract position identification module, and returns lesion identification result and the quality control operations to the capsule endoscopy data acquisition system.

Specifically, the processing flow of the digestive tract position identification module based on the image data and the sensor data includes: firstly, the image data screening module pre-processes the image data received from the capsule endoscopy data acquisition system to remove various unclear, over-bright or over-dark images; secondly, the digestive tract anatomical region identification algorithm identifies the anatomical region of the digestive tract according to the screened images, and the digestive tract lesion identification algorithm identifies the positive lesion in the digestive tract; thirdly, the position identification module identifies the relative position of the digestive tract where the capsule endoscope is located and the positive lesions at current position by analysis of anatomical region of the digestive tract and sensor data from sensors inside the capsule endoscope. The sensors inside the capsule endoscope comprise, but not limited to, the acceleration sensor, gyroscope, TOF distance sensor and magnetic field sensor.

The digestive tract position identification module employs a heterogeneous computing technology of CPU+GPU or CPU+FPGA in the image processing method. Refer to the above description for details, which are not described herein again.

The specific quality control process of the local server can be found in FIG. 2 and the foregoing description, and is simply described as follows: firstly, the digestive tract position identification module identifies the digestive tract position information and lesion information by processing the image data and the sensor data, and sends the identified information to the corresponding matching module for quality control process; secondly, the corresponding matching module for quality control process generates a corresponding quality control operation code according to the digestive tract position information, the lesion information and the operation quality control model, and transmits the quality control operation code to the capsule endoscopy data acquisition system, wherein the quality control operation code contains the information about the operation that should be performed currently; thirdly, after receiving the quality control operation code, the capsule endoscopy data acquisition system determines whether the capsule endoscope reaches the threshold of the digestive tract position segment, and if the threshold is reached, specific contents of the quality control operation code is presented to an operator; otherwise, a quality control identification is recorded.

Further, the capsule endoscopy image review and quality control method further comprises as quality control process: when the digestive tract position identification module identifies a region of the digestive tract, the region is highlighted on a simulated digestive tract 3D model on the display of the capsule endoscopy data acquisition system; when the digestive tract position identification module identifies a suspected lesion in the digestive tract, the quality control operation code is presented to the operator in the real-time browsing interface of the display.

The capsule endoscopy image review and quality control method further comprises an automatic configuration method and procedure of the local server and the capsule endoscopy data acquisition system, referring to FIG. 4 and foregoing description, wherein: the capsule endoscopy data acquisition system sends an IP multi-cast or IP broadcast message to the local area network, and waits for the unicast response from the local server; if the capsule endoscopy data acquisition system receives the message from the local server, the capsule endoscopy data acquisition system records the IP address of the local server and establishes Socket or RPC connection with the local server; after the connection is established, the system configurations of the capsule endoscopy data acquisition system and the local server are synchronized.

The capsule endoscopy image review and quality control method further comprises: remotely updating the main control program, image processing algorithm, the digestive tract anatomical region identification algorithm, the digestive tract lesion identification algorithm, and deep learning model for the local server through the cloud server.

The capsule endoscopy image review and quality control system and control method thereof disclosed herein generates the quality control operations according to the processing results of the digestive tract position identification module through the corresponding matching module for quality control process, and returns the lesion identification result and the quality control operations to the capsule endoscopy data acquisition system. It has guiding significance for the operation of physicians.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to affect such feature, structure, or characteristic in connection with other ones of the embodiments. Furthermore, for ease of understanding, certain method procedures may have been delineated as separate procedures; however, these separately delineated procedures should not be construed as necessarily order dependent in their performance. That is, some procedures may be able to be performed in an alternative ordering, simultaneously, etc. In addition, exemplary diagrams illustrate various methods in accordance with embodiments of the present disclosure. Such exemplary method embodiments are described herein using and can be applied to corresponding apparatus embodiments, however, the method embodiments are not intended to be limited thereby.

Although few embodiments of the present invention have been illustrated and described, it would be appreciated by those skilled in the art that changes may be made in these embodiments without departing from the principles and spirit of the invention. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein. As used in this disclosure, the term “preferably” is non-exclusive and means “preferably, but not limited to.” Terms in the claims should be given their broadest interpretation consistent with the general inventive concept as set forth in this description. For example, the terms “coupled” and “connect” (and derivations thereof) are used to connote both direct and indirect connections/couplings. As another example, “having” and “including”, derivatives thereof and similar transitional terms or phrases are used synonymously with “comprising” (i.e., all are considered “open ended” terms)—only the phrases “consisting of” and “consisting essentially of” should be considered as “close ended”. Claims are not intended to be interpreted under 112 sixth paragraph unless the phrase “means for” and an associated function appear in a claim and the claim fails to recite sufficient structure to perform such function.

1. A capsule endoscopy image review and quality control system, comprising a capsule endoscopy data acquisition system and a local server in communication with the capsule endoscopy data acquisition system, wherein
the capsule endoscopy data acquisition system comprises a capsule endoscope, an external magnetic field device for controlling the movement and/or rotation of the capsule endoscope, and a controller in communication with both the capsule endoscope and the external magnetic field device;
the local server comprises a digestive tract position identification module and a corresponding matching module for quality control process, wherein the corresponding matching module for quality control process, and the digestive tract position identification module are in communication with the controller.

2. The capsule endoscopy image review and quality control system of claim 1, wherein the digestive tract position identification module comprises an image data screening module, a digestive tract region identification module, a digestive tract lesion identification module and a position identification module, wherein
the image data screening module screens image data acquired from the capsule endoscopy data acquisition system to remove unclear, over-bright or over-dark images;
the digestive tract region identification module comprises a digestive tract anatomical region identification algorithm for identifying anatomical regions of the digestive tract according to the screened images;
the digestive tract lesion identification module comprises a digestive tract lesion identification algorithm for identifying positive lesions of the digestive tract;
the position identification module identifies the relative position of the digestive tract where the capsule endoscope is located and the lesions at current position by analysis of anatomical region of the digestive tract and data from sensors inside the capsule endoscope, and sensors inside the capsule endoscope comprise an acceleration sensor, a gyroscope, a TOF distance sensor and a magnetic field sensor.

3. The capsule endoscopy image review and quality control system of claim 1, wherein the corresponding matching module for quality control process comprises a preset operation quality control model corresponding to the digestive tract position and/or the lesion information.

4. The capsule endoscopy image review and quality control system of claim 1, wherein the local server and the capsule endoscopy data acquisition system are connected through a local area network, or are directly connected through a switch, a router, and a networking cable.

5. The capsule endoscopy image review and quality control system of claim 1, further comprising a cloud server in communication with the local server.

6. The capsule endoscopy image review and quality control system of claim 5, wherein the cloud server is connected to the local server via the Internet or the intranet.

7. The capsule endoscopy image review and quality control system of claim 6, wherein the connection between the cloud server and the local server is encrypted.

8. The capsule endoscopy image review and quality control system of claim 5, wherein the service architecture of the cloud server includes web service, application service, cloud storage, load balancing and message queue service and deep learning service cluster.

9. A capsule endoscopy image review and quality control method, comprising
transmitting image data and sensor data to a local server by a capsule endoscopy data acquisition system;
achieving quality control process by the local server based on the image data and the sensor data through a digestive tract position identification module of the local server and a corresponding matching module for quality control process, of the local server, wherein the digestive tract position identification module processes the image data and the sensor data, and the corresponding matching module for quality control process generates quality control operations according to the processing results of the digestive tract position identification module, and returns lesion identification results and the quality control operations to the capsule endoscopy data acquisition system.

10. The capsule endoscopy image review and quality control method of claim 9, wherein the process flow of the digestive tract position identification module on the image data and the sensor data comprising
performing pre-processes on the image data received from the capsule endoscopy data acquisition system to remove unclear, over-bright or over-dark images, by an image data screening module;
identifying anatomical regions of the digestive tract according to the screened images using a digestive tract anatomical region identification algorithm by digestive tract position identification module, and a digestive tract lesion identification algorithm of the digestive tract position identification module identifies positive lesions in the digestive tract;
identifying the relative position of the digestive tract where the capsule endoscope is located by position identification module of the digestive tract position identification module—and the positive lesions at current position by analysis of anatomical region of the digestive tract and sensor data from sensors inside the capsule endoscope, wherein the sensors inside the capsule endoscope comprises an acceleration sensor, a gyroscope, a TOF distance sensor and a magnetic field sensor.

11. The capsule endoscopy image review and quality control method of claim 9, wherein the digestive tract position identification module employs a heterogeneous computing technology of CPU+GPU or CPU+FPGA in the image processing method.

12. The capsule endoscopy image review and quality control method of claim 9, wherein the quality control process of the local server comprises
first, identifying the digestive tract position information and lesion information by through processing the image data and the sensor data by the digestive tract position identification module, and sending the identified information to the corresponding matching module for quality control process;
second,
generating a corresponding quality control operation code according to digestive tract position information, lesion information and an operation quality control model, by the corresponding matching module for quality control process, and
transmitting the quality control operation code to the capsule endoscopy data acquisition system, wherein the quality control operation code contains the information about the operation performed currently;
third, determining, after receiving the quality control operation code, by the capsule endoscopy data acquisition system, whether the capsule endoscope reaches a threshold of the digestive tract position interval, if the threshold is reached, specific contents of the quality control operation code is presented to an operator, and if the threshold is not reached, a quality control identification is recorded.

13. The capsule endoscopy image review and quality control method of claim 12,
wherein the quality control process further comprises
when the digestive tract position identification module identifies a region of the digestive tract, the region is highlighted on a simulated digestive tract 3D model on the display of the capsule endoscopy data acquisition system, and when the digestive tract position identification module identifies a suspected lesion in the digestive tract, a quality control operation code corresponding to the suspected lesion is presented in a real-time browsing interface of the display.

14. The capsule endoscopy image review and quality control method of claim 9, further comprising an automatic configuration method and procedure of the local server and the capsule endoscopy data acquisition system, wherein
the capsule endoscopy data acquisition system sends an IP multi-cast or IP broadcast message to the local area network, and waits for the unicast response from the local server;
if the capsule endoscopy data acquisition system receives message from the local server, the capsule endoscopy data acquisition system records the IP address of the local server and establishes Socket or RPC connection with the local server;
after the connection is established, the configurations of the capsule endoscopy data acquisition system and the local server are synchronized.

15. The capsule endoscopy image review and quality control method of claim 9, further comprising remotely updating main control program, image processing algorithm, anatomical region identification algorithm, digestive tract lesion identification algorithm, deep learning model for the local server through a cloud server.
